(19) United States
(12) Patent Application Publication
(10) Pub. No.: US 2012/0290043 A1
(43) Pub. Date: Nov. 15, 2012

(54) IMPLANTED ENERGY SOURCE

(75) Inventor: Yossi GROSS, Moshav Mazor (IL)

(73) Assignee: RAINBOW MEDICAL LTD., HERZLIYA (IL)

(21) Appl. No.: 13/106,502

(22) Filed: May 12, 2011

(51) Int. Cl. A61N 1/36

(52) U.S. Cl. 607/48

(57) ABSTRACT

Apparatus and methods are described for use with (a) a muscle of a body of a subject and (b) an implanted functional device. An electrode is electrically coupled to the muscle. A compressible housing is compressed due to the movement of the muscle. An energy assembly includes a flywheel and an energy converter. The energy assembly (a) stores energy extracted from the movement of the muscle, by rotating the flywheel, (b) causes the muscle to undergo movement by driving a current into the muscle, via the electrode, using the stored energy, and (c) powers the implanted functional device using the stored energy. A chamber, in fluid communication with the housing, receives, from the compressible housing, fluid that is expelled from the compressible housing due to the compression of the compressible housing. Other applications are also described.

### CROSS REFERENCES TO RELATED APPLICATIONS

The present patent application is related to U.S. patent application Ser. No. 12/153,039 (published as US 2008/0262567 to Avrahami), filed on May 13, 2008, entitled “Implanted energy source,” which is a continuation-in-part of U.S. patent application Ser. No. 10/891,156, filed on Jul. 15, 2004, entitled “Converting biomechanical energy to electrical/mechanical energy” (published as US 2005/0027332 to Avrahami, entitled “Implanted autonomic energy source”). Both of the aforementioned references are incorporated herein by reference.

### FIELD OF EMBODIMENTS OF THE INVENTION

Some applications of the present invention generally relate to implantable medical apparatus. Specifically, some applications of the present invention relate to implantable energy sources.

### BACKGROUND

Implantable functional devices that consume energy are well known. For example, implantable devices are used for sensing and controlling physiological parameters, tissue stimulation, drug dispensing, external communication, and to perform functions such as heart assistance, pumping, and neurological stimulation.

### SUMMARY OF EMBODIMENTS

For some applications of the present invention, at least one electrode is coupled to a muscle of a subject's body. An energy assembly causes the muscle to undergo movement by driving a current into the muscle, via the electrode. The energy assembly extracts energy from the movement of the muscle, and powers both the driving of the current via the electrode and an implanted functional device using the extracted energy.

For some applications, the energy assembly includes a flywheel and an energy converter (e.g., a generator). The flywheel stores extracted energy associated with the movement of the muscle by being rotated due to the movement of the muscle. Based on the movement of the muscle, the energy converter powers (a) the driving of the current via the electrode, and (b) the implanted functional device. For example, the energy converter may convert to electrical energy, kinetic energy associated with the rotation of the flywheel. A first portion of the generated electrical energy is used to power the driving of the current via the electrode, and a second portion of the generated electricity is used to power the implanted functional device. For some applications, the energy assembly is configured such that movement of the flywheel does not result in substantial changes in the pressure of fluid (e.g., air) that is disposed inside a chamber in which the flywheel is disposed.

For some applications, the energy assembly includes two arms that are pivotally coupled with one another at a pivot and that are placed with respect to the muscle such that the arms pivot with respect to one another due to the movement of the muscle. An energy converter (e.g., a generator) disposed at the pivot utilizes energy extracted due to the movement of the muscle. Based on the movement of the muscle, the energy converter powers (a) the driving of the current via the electrode, and (b) the implanted functional device. For example, the energy converter may convert to electrical energy, kinetic energy associated with the pivoting of the arms with respect to one another. A first portion of the generated electrical energy is used to power the driving of the current via the electrodes, and a second portion of the generated electricity is used to power the implanted functional device.

There is therefore provided, in accordance with some applications of the present invention, apparatus for use with (a) a muscle of a body of a subject and (b) an implanted functional device in the subject's body, the apparatus including:

an electrode configured to be electrically coupled to the muscle;

a compressible housing configured to become compressed due to the movement of the muscle;

an energy assembly including a flywheel and an energy converter, the energy assembly being configured:
to store energy extracted from the movement of the muscle, by rotating the flywheel,
to cause the muscle to undergo movement by driving a current into the muscle, via the electrode, using the stored energy, and
to power the implanted functional device using the stored energy; and

a chamber in fluid communication with the housing and configured to receive, from the compressible housing, fluid that is expelled from the compressible housing due to the compression of the compressible housing.

For some applications, the energy converter includes a coil and magnet, and the energy converter is configured to generate electrical energy due to the compression of the housing, by moving the coil with respect to the magnet.

For some applications, the energy converter includes a piezoelectric element, and the energy converter is configured to generate electrical energy due to the compression of the housing, by causing the piezoelectric element to become compressed.

For some applications, the chamber is in contact with the compressible housing.

For some applications, the apparatus further includes a tube disposed between the compressible housing and the chamber, and configured to provide fluid communication between the compressible housing and the chamber.

For some applications, the chamber is placed in fluid communication with the compressible housing such that fluid inside the housing does not undergo a substantial increase in pressure due to the compression of the compressible housing.

For some applications, the chamber is placed in fluid communication with the compressible housing such that fluid inside the housing undergoes an increase in pressure of less than 0.3 atm due to the compression of the compressible housing.

For some applications, the chamber is placed in fluid communication with the compressible housing such that fluid inside the housing undergoes an increase in pressure of less than 0.2 atm due to the compression of the compressible housing.

There is additionally provided, in accordance with some applications of the present invention, apparatus for use with (a) a muscle of a body of a subject and (b) an implanted functional device in the subject's body, the apparatus including:

an electrode configured to be electrically coupled to the muscle;

a rigid housing; and

an energy assembly disposed inside the rigid housing, the energy assembly including a flywheel and an energy converter, the energy assembly being configured:
to store energy extracted from the movement of the muscle, by rotating the flywheel,
to cause the muscle to undergo movement by driving a current into the muscle, via the electrode, using the stored energy, and
to power the implanted functional device using the stored energy.

For some applications, the housing is configured not to become compressed due to the movement of the muscle.

For some applications, the housing is configured such that fluid inside the housing does not undergo a substantial increase in pressure due to the movement of the muscle.

For some applications, the energy converter includes a coil and magnet, and the energy converter is configured to generate electrical energy due to the movement of the muscle, by moving the coil with respect to the magnet.

For some applications, the energy converter includes a piezoelectric element, and the energy converter is configured to generate electrical energy due to the movement of the muscle, by causing the piezoelectric element to become compressed.

There is additionally provided, in accordance with some applications of the present invention, apparatus for use with (a) a muscle of a body of a subject and (b) an implanted functional device in the subject's body, the apparatus including:

an electrode configured to be electrically coupled to the muscle;

a housing; and

an energy assembly disposed inside the housing, the energy assembly including a flywheel and an energy converter, the energy assembly being configured:
to store energy extracted from the movement of the muscle by rotating the flywheel,
to cause the muscle to undergo movement by driving a current into the muscle, via the electrode, using the stored energy, and
to power the implanted functional device using the stored energy,

the housing being configured not to become compressed due to the movement of the muscle.

For some applications, the housing is rigid.

For some applications, the housing is configured such that fluid inside the housing does not undergo a substantial increase in pressure due to the movement of the muscle.

For some applications, the energy converter includes a coil and magnet, and the energy converter is configured to generate electrical energy due the movement of the muscle, by moving the coil with respect to the magnet.

For some applications, the energy converter includes a piezoelectric element, and the energy converter is configured to generate electrical energy due to the movement of the muscle by causing the piezoelectric element to become compressed.

There is further provided, in accordance with some applications of the present invention, apparatus for use with (a) a muscle of a body of a subject and (b) an implanted functional device in the subject's body, the apparatus including:

an electrode configured to be electrically coupled to the muscle;

two arms that are pivotally coupled with one another at a pivot, and that are configured to be disposed with respect to the muscle such that the arms pivot with respect to one another due to the movement of the muscle; and

an energy converter, configured to:
extract energy from the pivoting of the arms,
cause the muscle to undergo movement by driving a current into the muscle, via the electrode, using the extracted energy, and
power the implanted functional device using the extracted energy.

For some applications, the energy converter includes a coil and magnet, and the energy converter is configured to extract electrical energy from the pivoting of the arms by moving the coil with respect to the magnet.

For some applications, the energy converter includes a piezoelectric element, and the energy converter is configured to extract electrical energy from the pivoting of the arms by causing the piezoelectric element to become compressed.

For some applications, the apparatus further includes a generally rigid housing, the arms being disposed inside the housing.

For some applications, the housing is configured such that fluid inside the housing does not undergo a substantial increase in pressure due to the movement of the muscle.

There is additionally provided, in accordance with some applications of the present invention, a method for use with (a) a muscle of a body of a subject and (b) an implanted functional device in the subject's body, the method including:

electrically coupling an electrode to the muscle;

placing in a vicinity of the muscle a compressible housing such that the compressible housing becomes compressed due to the movement of the muscle;

storing energy from the compression of the housing by rotating a flywheel disposed inside the housing;

causing the muscle to undergo movement by driving a current into the muscle, via the electrode, using the stored energy; and

powering an implanted functional device using the stored energy; and

receiving into a chamber that is in fluid communication with the housing, fluid that is expelled from the compressible housing due to the compression of the compressible housing.

For some applications, storing energy includes extracting electrical energy by moving a coil with respect to a magnet.

For some applications, storing energy from the compression of the housing includes extracting electrical energy by causing a piezoelectric element to become compressed.

For some applications, receiving the fluid into the chamber includes receiving the fluid into a chamber that is in contact with to the compressible housing.

For some applications, receiving the fluid into the chamber includes receiving the fluid into a chamber that is disposed remotely from the compressible housing.

For some applications, receiving the fluid into the chamber includes reducing an increase in pressure undergone by fluid inside the housing due to the compression of the housing, relative to an increase in pressure undergone by fluid inside the housing due to the compression of the housing in the absence of the chamber.

For some applications, receiving the fluid into the chamber includes reducing the increase in pressure undergone by fluid inside the housing due to the compression of the housing to less than 0.3 atm.

For some applications, receiving the fluid into the chamber includes reducing the increase in pressure undergone by fluid inside the housing due to the compression of the housing to less than 0.2 atm.

There is additionally provided, in accordance with some applications of the present invention, a method for use with (a) a muscle of a body of a subject and (b) an implanted functional device in the subject's body, the method including:

electrically coupling an electrode to the muscle;

placing a flywheel in a vicinity of the muscle such that the flywheel is rotated due to movement of the muscle, the flywheel being disposed inside a housing;

storing energy from the movement of the muscle by rotating the flywheel, without compressing the housing;

causing the muscle to undergo movement by driving a current into the muscle, via the electrode, using the stored energy; and

powering the implanted functional device using the stored energy.

For some applications, placing the flywheel in the vicinity of the muscle includes placing the flywheel in the vicinity of the muscle, the flywheel being disposed inside a rigid housing.

For some applications, storing the energy includes extracting electrical energy by moving a coil with respect to a magnet.

For some applications, storing the energy includes extracting electrical energy by causing a piezoelectric element to become compressed.

There is additionally provided, in accordance with some applications of the present invention, a method for use with (a) a muscle of a body of a subject and (b) an implanted functional device in the subject's body, the method including:

electrically coupling an electrode to the muscle;

placing in a vicinity of the muscle, two arms that are pivotally coupled with one another at a pivot, such that the arms pivot with respect to one another due to the movement of the muscle;

extracting energy from the pivoting of the arms;

causing the muscle to undergo movement by driving a current into the muscle, via the electrode, using the extracted energy; and

powering the implanted functional device using the extracted energy.

For some applications, extracting the energy includes extracting electrical energy by moving the coil with respect to the magnet.

For some applications, extracting the energy includes extracting electrical energy by causing a piezoelectric element to become compressed.

There is further provided, in accordance with some applications of the present invention, apparatus for use with (a) a muscle of a body of a subject and (b) an implanted functional device in the subject's body, the apparatus including:

an electrode configured to be electrically coupled to the muscle;

a chamber that contains a fluid;

an energy-extraction device configured to extract energy by being moved by the movement of the muscle;

a generator configured to generate electrical energy from the extracted energy and to use the generated energy to drive a current into the muscle via the electrode, and to power the implanted functional device,

the energy extraction-device being disposed inside the chamber, and

the chamber being configured such that a pressure of the fluid inside the chamber does not increase by more than 0.3 atm, during the extraction of the energy by the energy-extraction device.

For some applications, the chamber is configured such that a pressure of the fluid inside the chamber does not increase by more than 0.2 atm, during the extraction of the energy by the energy-extraction device.

The present invention will be more fully understood from the following detailed description of embodiments thereof, taken together with the drawings, in which:

### BRIEF DESCRIPTION OF THE DRAWINGS

FIGS. 1A-B are schematic illustrations of an implantable flywheel for providing energy to an implanted functional device, in accordance with some applications of the present invention;

FIG. 2 is a schematic illustration of the flywheel of FIGS. 1A-B, the flywheel having been implanted inside chest-muscles of the subject, in accordance with some applications of the present invention;

FIG. 3 is a schematic illustration of a flywheel structure disposed inside a rigid housing, in accordance with some applications of the present invention;

FIG. 4A is a schematic illustration of a flywheel housing that is in fluid communication with a chamber, in accordance with some applications of the present invention;

FIG. 4B is a schematic illustration of a flywheel housing that is in fluid communication with a remote chamber, in accordance with some applications of the present invention; and

FIGS. 5-8D are schematic illustrations of respective views of a device that defines two arms that are pivotally coupled to one another for implanting in a vicinity of a muscle, in accordance with some applications of the present invention.

### DETAILED DESCRIPTION OF EMBODIMENTS

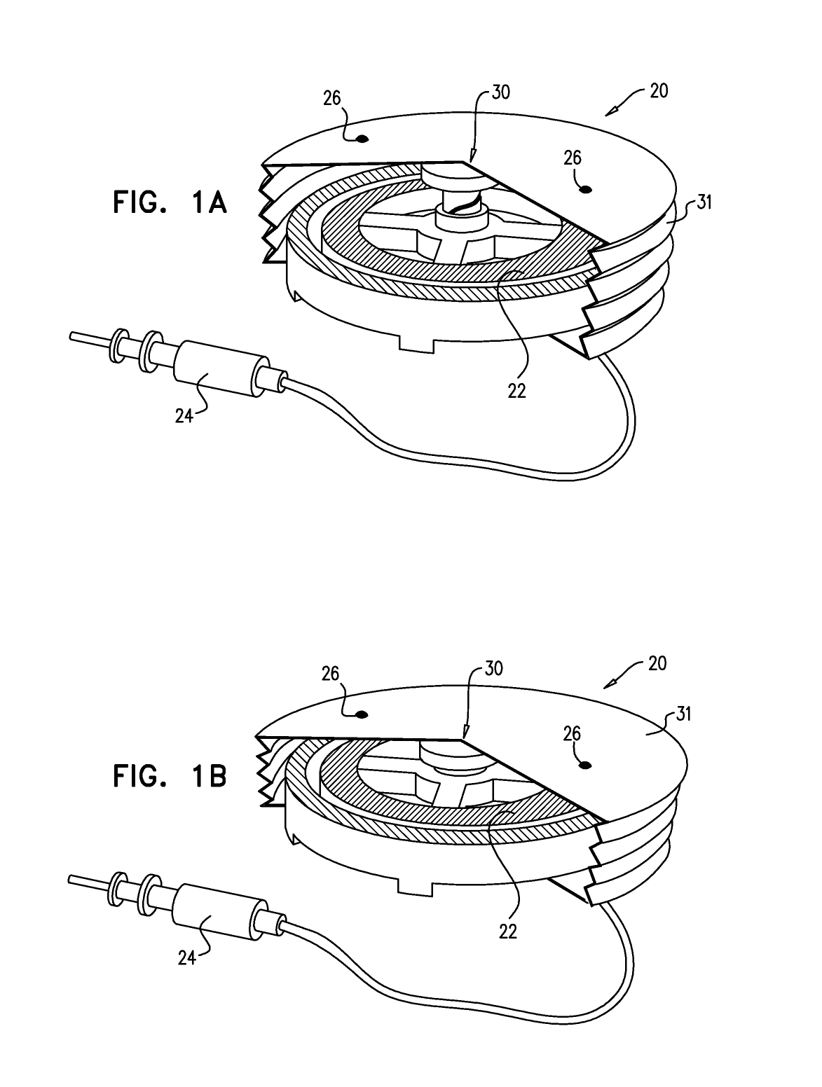

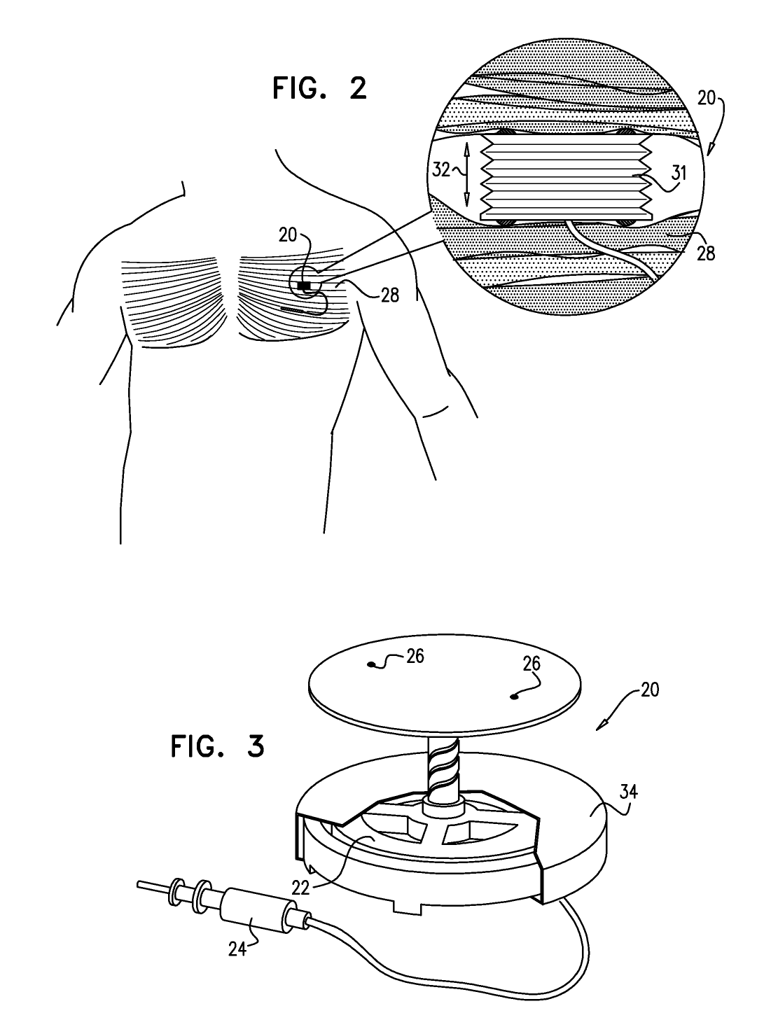

Reference is now made to FIGS. 1-2, which are schematic illustrations of an implantable energy assembly 20 (which for applications shown in FIGS. 1-4 includes a flywheel 22), for providing energy to an implanted functional device 24, in accordance with some applications of the present invention. Typically, energy assembly 20 effects an energy loop, generally in accordance with the techniques described in US 2008/0262567 to Avrahami, which is incorporated herein by reference. At least one electrode 26 (e.g., two electrodes, as shown) is typically placed in contact with a muscle 28 of a subject. A current is driven into the muscle via the electrodes, causing the muscle to move. Energy assembly 20 converts energy associated with the movement of the muscle (e.g., from kinetic energy into electrical energy), and using this energy, powers both implanted functional device 24 and the driving of the current via electrodes 26.

For some applications, flywheel 22 stores energy extracted due to the movement of the muscle. An energy converter 30 (e.g., a generator) powers the implanted functional device 24 and the driving of the current via electrodes 26, based on the extracted energy, by converting the extracted energy to electrical energy. For some applications, the energy assembly powers the stimulator and/or the implanted functional device using techniques described in US 2008/0262567 to Avrahami and/or US 2005/0027332 to Avrahami, both of which applications are incorporated herein by reference. For example, the energy assembly may include an energy converter that generates power by moving a coil through a magnetic field of a magnet (or vice versa), in order to generate electricity, and/or by applying a force to a piezoelectric element. Typically, rotation of the flywheel due to the movement of the muscle is used to generate electrical power that is used to power (a) the driving of the current via electrodes 26 and (b) implanted functional device 24.

Typically, each time energy assembly 20 stimulates muscle 28 to move (by driving a current into the muscle via electrodes 26), the energy assembly extracts an amount of energy from the resultant movement of the muscle, that exceeds the amount of energy that was used by the energy assembly in order to stimulate the muscle. At least a portion of the excess energy is used to power implanted functional device 24.

For some applications, implanted functional device 24 is configured to sense a parameter of the subject's body, control a parameter of the subject's body, stimulate tissue, dispense a drug, communicate with a device that is external to the patient's body, assist cardiac function, act as a pump, and/or stimulate neurological functions of the subject.

For some applications, electrodes 26 are not driven to drive a current into the subject's muscle continuously. For example, the current may be driven for only a given period of time followed by a rest period during each 24-hour period, for only a given period of time followed by a rest period each hour, and/or for only a given period of time followed by a rest period each minute. For some applications, muscle fatigue is reduced by not driving the current into the muscle continuously, relative to if the current were driven into the muscle continuously. For some applications, the current is driven into the muscle continuously (in this context, at least once during every minute).

For some applications, as shown in FIGS. 1-2, flywheel 22 is disposed inside a compressible housing 31 that becomes compressed and uncompressed due to movement of muscle 28, as indicated by arrow 32, shown in FIG. 2.

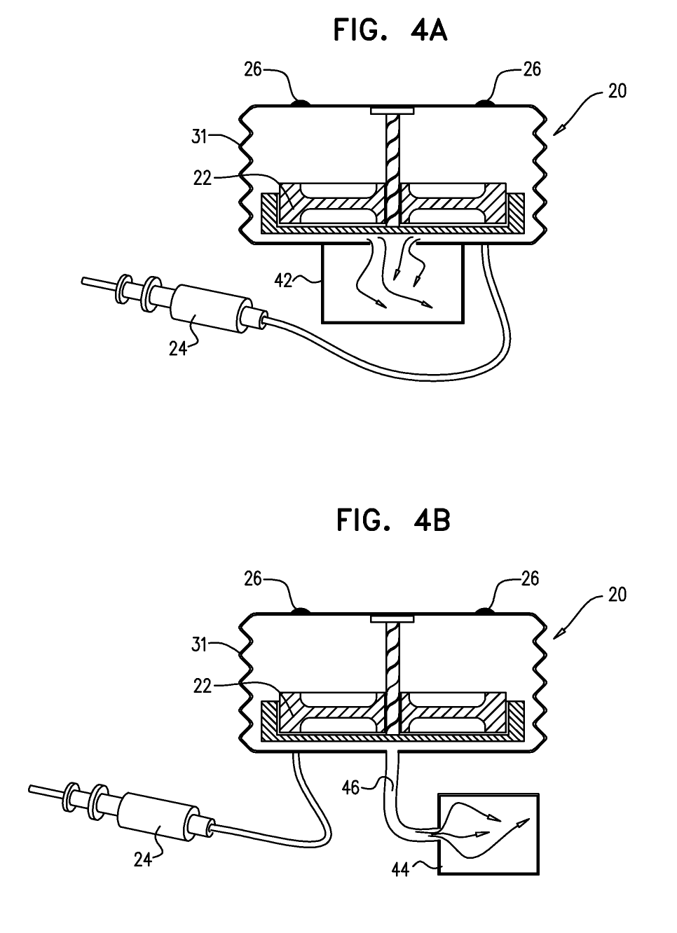

Reference is now made to FIG. 3, which is a schematic illustration of implantable energy assembly 20 including a flywheel 22 disposed inside a rigid housing 34, in accordance with some applications of the present invention. The general techniques described with reference to FIGS. 1-2 are used in conjunction with energy assembly 20 shown in FIG. 3. However, whereas in the energy assembly shown in FIGS. 1-2, flywheel 22 is disposed inside compressible housing 31, in the energy assembly shown in FIG. 3, flywheel is disposed inside rigid housing 34. For some applications, due to the rigidity of housing 34, housing 34 does not become compressed when muscle 28 (shown in FIG. 2) moves and compresses the energy assembly. For some applications, the movement of the muscle does not cause fluid (e.g., air) inside housing 34 to undergo a substantial change in pressure, since the rigidity of the housing prevents the fluid inside the housing from becoming compressed. For example, the fluid may undergo a pressure change of less than 0.3 atm, e.g., less than 0.2 atm, in response to the muscle compressing the energy assembly.

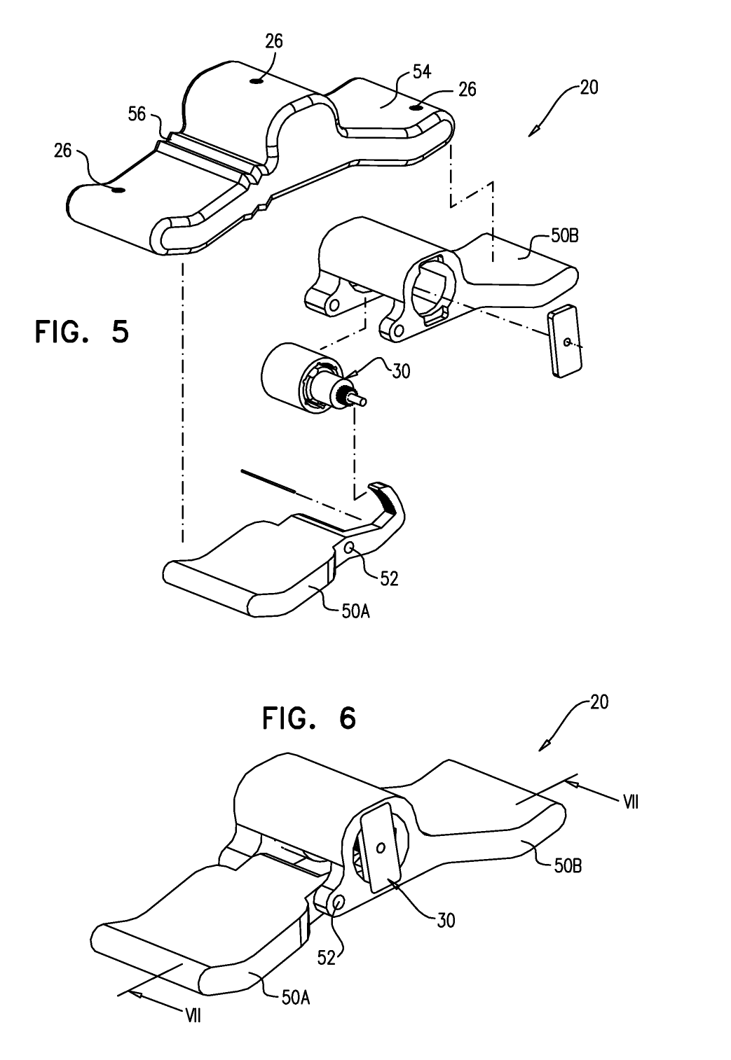

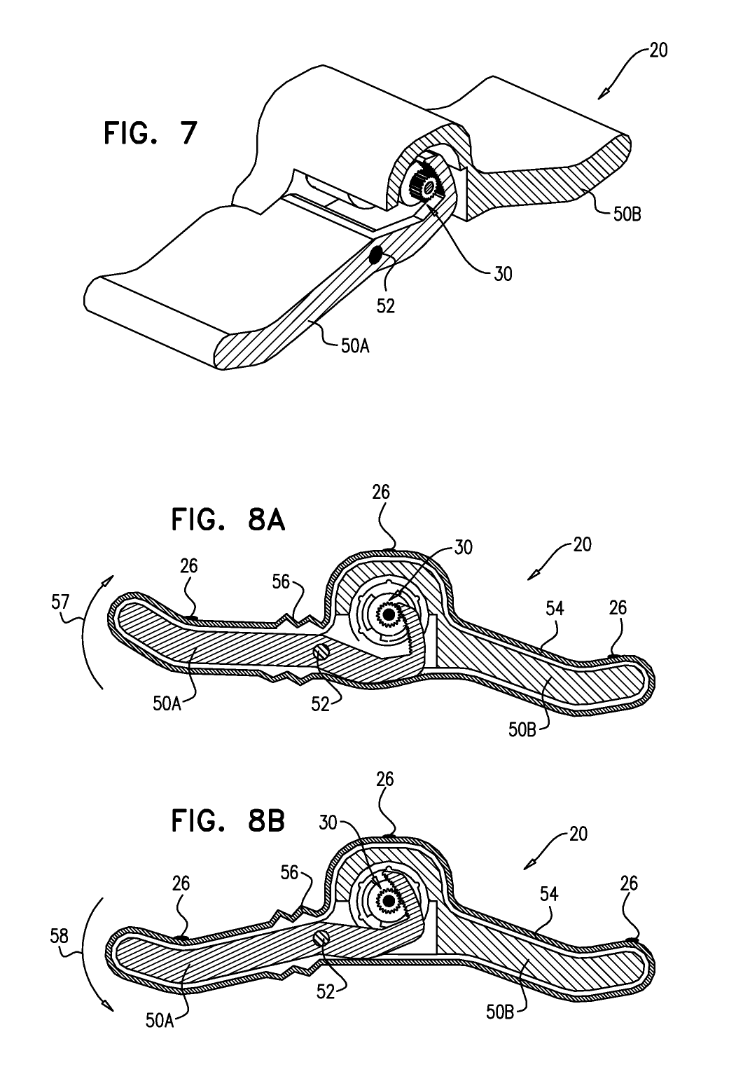

Reference is now made to FIG. 4A, which is a schematic illustration of implantable energy assembly 20, including a flywheel 22 disposed inside compressible housing 31, housing 31 being in contact with and in fluid communication with an adjacent chamber 42, in accordance with some applications of the present invention. The general techniques described with reference to FIGS. 1-2 are used in conjunction with energy assembly 20 shown in FIG. 3. In the energy assembly shown in FIGS. 1-2, movement of the muscle (and the resultant compression of the compressible housing) may cause fluid inside housing 31 to undergo an increase in pressure. In the energy assembly shown in FIG. 4A, in response to housing 31 becoming compressed, at least some of the fluid inside the housing enters adjacent chamber 42. For some applications, the transfer of fluid from compressible housing 31 to chamber 42 reduces or prevents a pressure increase inside housing 40 that may have resulted from the compression of housing 31, in the absence of adjacent chamber 42. For some applications, the pressure inside housing 31 increases by less than 0.3 atm, e.g., less than 0.2 atm, as a result of the compression of the housing.

For some applications, chamber 42 is compressible, e.g., having a bellows configuration. Typically, but not necessarily, the volume of adjacent chamber 42 is more than 0.5 times and/or less than twice (e.g., approximately equal to) the volume of fluid that is passed out of compressible housing 31 during each cycle of compression and uncompression of compressible housing 31.

Reference is now made to FIG. 4B, which is a schematic illustration of implantable energy assembly 20, including a flywheel 22 disposed inside compressible housing 31 that is in fluid communication with a remote chamber 44, in accordance with some applications of the present invention. Energy assembly 20 shown in FIG. 4B is generally similar to the energy assembly shown in FIG. 4A. However, in accordance with some applications, in the energy assembly shown in FIG. 4B, the chamber into which fluid from compressible housing 31 enters, is disposed remotely from the compressible housing. The remote chamber is in fluid communication with the compressible housing via a tube 46. For some applications, remote chamber 44 is used if it is not possible, or if it is difficult, to place both compressible chamber 31 and adjacent chamber 42 (shown in FIG. 4A) in the vicinity of the muscle. For some applications, chamber 44 is compressible, e.g., having a bellows configuration.

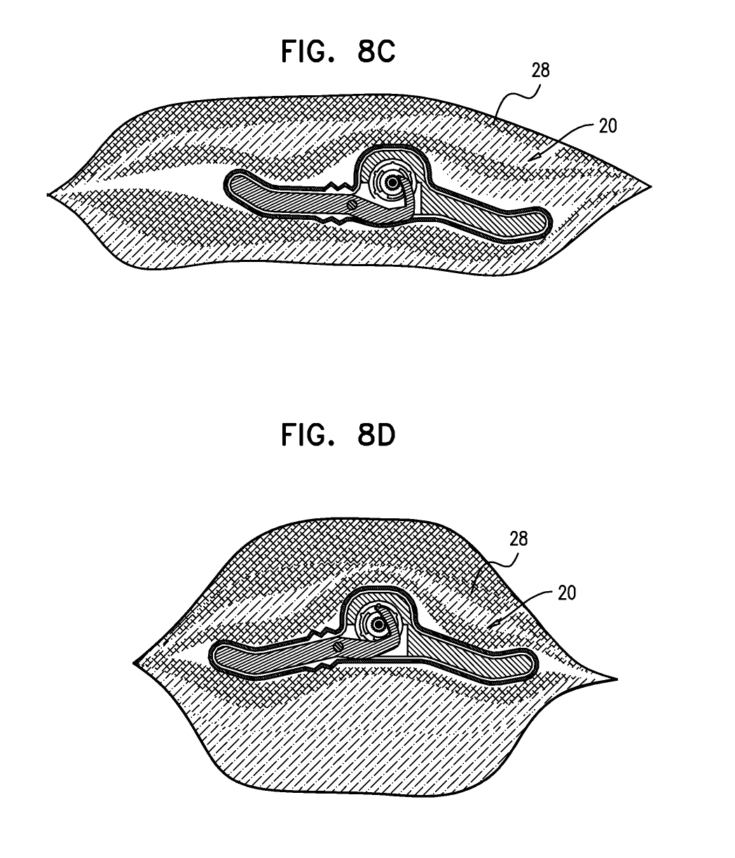

Reference is now made to FIGS. 5-8D, which are schematic illustrations of respective views of energy assembly 20, the energy assembly defining two arms 50A and 50B that are pivotally coupled to one another at a pivot 52, for implanting in a vicinity of muscle 28, in accordance with some applications of the present invention. Energy assembly 20 described with reference to FIGS. 5-8D is generally similar to energy assembly 20 described with reference to FIGS. 1-4B except for the differences described hereinbelow.

Arms 50A and 50B and pivot 52 are typically disposed inside a housing 54. Housing 54 is typically rigid, except for a flexible portion 56 of the housing. FIGS. 8C-D show energy assembly 20 disposed in a vicinity of muscle 28 (e.g., inside the muscle, as shown). When the muscle changes from a relaxed state (FIG. 8C) to a contracted state (FIG. 8D) and vice versa, arms 50A and 50B rotate with respect to one another about pivot 52. The rotation of the arms with respect to each other is also shown in FIGS. 8A-B. As shown in FIG. 8A, when the muscle undergoes a transition from a contracted state to a relaxed state, arm 50A rotates in the direction of arrow 57, with respect to arm 50B. As shown in FIG. 8B, when the muscle undergoes a transition from the relaxed state to the contracted state, arm 50A rotates in the direction of arrow 58 (i.e., the reverse of the direction arrow 57), with respect to arm 50B.

At least one electrode 26 is typically disposed on the outside of housing 54, such that the electrode is in contact with muscle 28. Electrodes are shown as being disposed on housing 54 above arms 50A and 50B, and above a central portion of the device. However, for some applications, the one or more electrodes are disposed at only one of the aforementioned locations, and/or at a different location on housing 54 (e.g., on the tip of one or both of the arms).

Energy converter 30 is typically disposed inside housing 54 (e.g., in the vicinity of pivot 52, as shown). As described hereinabove, the energy converter (e.g., a generator) powers (a) the driving of a current via electrodes 26, and (b) implanted functional device 24, based on the movement of the muscle. For example, the energy assembly may generate power by moving a coil through a magnetic field of a magnet (or vice versa) in order to generate electricity, and/or by applying a force to a piezoelectric element. Typically, movement of arms 50A and 50B with respect to one another due to the movement of the muscle is used to generate electrical power that is used to power (a) the driving of the current via electrodes 26 and (b) implanted functional device 24.

It is noted that although muscle 28 is shown in FIG. 2 as being a chest muscle of the subject, the scope of the present invention includes implanting energy assembly 20 in any suitable muscle, for example, an arm muscle, a leg muscle, and/or a muscle of the back, or of the shoulder.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with (a) a muscle of a body of a subject and (b) an implanted functional device in the subject's body, the apparatus comprising:
an electrode configured to be electrically coupled to the muscle;
a compressible housing configured to become compressed due to the movement of the muscle;
an energy assembly comprising a flywheel and an energy converter, the energy assembly being configured:
to store energy extracted from the movement of the muscle, by rotating the flywheel,
to cause the muscle to undergo movement by driving a current into the muscle, via the electrode, using the stored energy, and
to power the implanted functional device using the stored energy; and
a chamber in fluid communication with the housing and configured to receive, from the compressible housing, fluid that is expelled from the compressible housing due to the compression of the compressible housing.

2. The apparatus according to claim 1, wherein the energy converter comprises a coil and magnet, and wherein the energy converter is configured to generate electrical energy due to the compression of the housing, by moving the coil with respect to the magnet.

3. The apparatus according to claim 1, wherein the energy converter comprises a piezoelectric element, and wherein the energy converter is configured to generate electrical energy due to the compression of the housing, by causing the piezoelectric element to become compressed.

4. The apparatus according to claim 1, wherein the chamber is in contact with the compressible housing.

5. The apparatus according to claim 1, further comprising a tube disposed between the compressible housing and the chamber, and configured to provide fluid communication between the compressible housing and the chamber.

6. The apparatus according to claim 1, wherein the chamber is placed in fluid communication with the compressible housing such that fluid inside the housing does not undergo a substantial increase in pressure due to the compression of the compressible housing.

7. The apparatus according to claim 6, wherein the chamber is placed in fluid communication with the compressible housing such that fluid inside the housing undergoes an increase in pressure of less than 0.3 atm due to the compression of the compressible housing.

8. The apparatus according to claim 7, wherein the chamber is placed in fluid communication with the compressible housing such that fluid inside the housing undergoes an increase in pressure of less than 0.2 atm due to the compression of the compressible housing.

9. Apparatus for use with (a) a muscle of a body of a subject and (b) an implanted functional device in the subject's body, the apparatus comprising:
an electrode configured to be electrically coupled to the muscle;
a rigid housing; and
an energy assembly disposed inside the rigid housing, the energy assembly comprising a flywheel and an energy converter, the energy assembly being configured:
to store energy extracted from the movement of the muscle, by rotating the flywheel,
to cause the muscle to undergo movement by driving a current into the muscle, via the electrode, using the stored energy, and
to power the implanted functional device using the stored energy.

10. The apparatus according to claim 9, wherein the housing is configured not to become compressed due to the movement of the muscle.

11. The apparatus according to claim 9, wherein the housing is configured such that fluid inside the housing does not undergo a substantial increase in pressure due to the movement of the muscle.

12. The apparatus according to claim 9, wherein the energy converter comprises a coil and magnet, and wherein the energy converter is configured to generate electrical energy due to the movement of the muscle, by moving the coil with respect to the magnet.

13. The apparatus according to claim 9, wherein the energy converter comprises a piezoelectric element, and wherein the energy converter is configured to generate electrical energy due to the movement of the muscle, by causing the piezoelectric element to become compressed.

14. Apparatus for use with (a) a muscle of a body of a subject and (b) an implanted functional device in the subject's body, the apparatus comprising:
an electrode configured to be electrically coupled to the muscle;
a housing; and
an energy assembly disposed inside the housing, the energy assembly comprising a flywheel and an energy converter, the energy assembly being configured:
to store energy extracted from the movement of the muscle by rotating the flywheel,
to cause the muscle to undergo movement by driving a current into the muscle, via the electrode, using the stored energy, and
to power the implanted functional device using the stored energy,
the housing being configured not to become compressed due to the movement of the muscle.

15. The apparatus according to claim 14, wherein the housing is rigid.

16. The apparatus according to claim 14, wherein the housing is configured such that fluid inside the housing does not undergo a substantial increase in pressure due to the movement of the muscle.

17. The apparatus according to claim 14, wherein the energy converter comprises a coil and magnet, and wherein the energy converter is configured to generate electrical energy due the movement of the muscle, by moving the coil with respect to the magnet.

18. The apparatus according to claim 14, wherein the energy converter comprises a piezoelectric element, and wherein the energy converter is configured to generate electrical energy due to the movement of the muscle by causing the piezoelectric element to become compressed.

19. Apparatus for use with (a) a muscle of a body of a subject and (b) an implanted functional device in the subject's body, the apparatus comprising:
an electrode configured to be electrically coupled to the muscle;
two arms that are pivotally coupled with one another at a pivot, and that are configured to be disposed with respect to the muscle such that the arms pivot with respect to one another due to the movement of the muscle; and
an energy converter, configured to:
extract energy from the pivoting of the arms,
cause the muscle to undergo movement by driving a current into the muscle, via the electrode, using the extracted energy, and
power the implanted functional device using the extracted energy.

20. The apparatus according to claim 19, wherein the energy converter comprises a coil and magnet, and wherein the energy converter is configured to extract electrical energy from the pivoting of the arms by moving the coil with respect to the magnet.

21. The apparatus according to claim 19, wherein the energy converter comprises a piezoelectric element, and wherein the energy converter is configured to extract electrical energy from the pivoting of the arms by causing the piezoelectric element to become compressed.

22. The apparatus according to claim 19, further comprising a generally rigid housing, the arms being disposed inside the housing.

23. The apparatus according to claim 22, wherein the housing is configured such that fluid inside the housing does not undergo a substantial increase in pressure due to the movement of the muscle.

24. A method for use with (a) a muscle of a body of a subject and (b) an implanted functional device in the subject's body, the method comprising:
electrically coupling an electrode to the muscle;
placing in a vicinity of the muscle a compressible housing such that the compressible housing becomes compressed due to the movement of the muscle;
storing energy from the compression of the housing by rotating a flywheel disposed inside the housing;
causing the muscle to undergo movement by driving a current into the muscle, via the electrode, using the stored energy; and
powering an implanted functional device using the stored energy; and
receiving into a chamber that is in fluid communication with the housing, fluid that is expelled from the compressible housing due to the compression of the compressible housing.

25. The method according to claim 24, wherein storing energy comprises extracting electrical energy by moving a coil with respect to a magnet.

26. The method according to claim 24, wherein storing energy from the compression of the housing comprises extracting electrical energy by causing a piezoelectric element to become compressed.

27. The method according to claim 24, wherein receiving the fluid into the chamber comprises receiving the fluid into a chamber that is in contact with to the compressible housing.

28. The method according to claim 24, wherein receiving the fluid into the chamber comprises receiving the fluid into a chamber that is disposed remotely from the compressible housing.

29. The method according to claim 24, wherein receiving the fluid into the chamber comprises reducing an increase in pressure undergone by fluid inside the housing due to the compression of the housing, relative to an increase in pressure undergone by fluid inside the housing due to the compression of the housing, in the absence of the chamber.

30. The method according to claim 29, wherein receiving the fluid into the chamber comprises reducing the increase in pressure undergone by fluid inside the housing due to the compression of the housing to less than 0.3 atm.

31. The method according to claim 30, wherein receiving the fluid into the chamber comprises reducing the increase in pressure undergone by fluid inside the housing due to the compression of the housing to less than 0.2 atm.

32. A method for use with (a) a muscle of a body of a subject and (b) an implanted functional device in the subject's body, the method comprising:
electrically coupling an electrode to the muscle;
placing a flywheel in a vicinity of the muscle such that the flywheel is rotated due to movement of the muscle, the flywheel being disposed inside a housing;
storing energy from the movement of the muscle by rotating the flywheel, without compressing the housing;
causing the muscle to undergo movement by driving a current into the muscle, via the electrode, using the stored energy; and
powering the implanted functional device using the stored energy.

33. The method according to claim 32, wherein placing the flywheel in the vicinity of the muscle comprises placing the flywheel in the vicinity of the muscle, the flywheel being disposed inside a rigid housing.

34. The method according to claim 32, wherein storing the energy comprises extracting electrical energy by moving a coil with respect to a magnet.

35. The method according to claim 32, wherein storing the energy comprises extracting electrical energy by causing a piezoelectric element to become compressed.

36. A method for use with (a) a muscle of a body of a subject and (b) an implanted functional device in the subject's body, the method comprising:
electrically coupling an electrode to the muscle;
placing in a vicinity of the muscle, two arms that are pivotally coupled with one another at a pivot, such that the arms pivot with respect to one another due to the movement of the muscle;
extracting energy from the pivoting of the arms;
causing the muscle to undergo movement by driving a current into the muscle, via the electrode, using the extracted energy; and
powering the implanted functional device using the extracted energy.

37. The method according to claim 36, wherein extracting the energy comprises extracting electrical energy by moving the coil with respect to the magnet.

38. The method according to claim 36, wherein extracting the energy comprises extracting electrical energy by causing a piezoelectric element to become compressed.

39. Apparatus for use with (a) a muscle of a body of a subject and (b) an implanted functional device in the subject's body, the apparatus comprising:
an electrode configured to be electrically coupled to the muscle;
a chamber that contains a fluid;
an energy-extraction device configured to extract energy by being moved by the movement of the muscle;
a generator configured to generate electrical energy from the extracted energy and to use the generated energy to drive a current into the muscle via the electrode, and to power the implanted functional device,
the energy extraction-device being disposed inside the chamber, and
the chamber being configured such that a pressure of the fluid inside the chamber does not increase by more than 0.3 atm, during the extraction of the energy by the energy-extraction device.

40. The apparatus according to claim 39, wherein the chamber is configured such that a pressure of the fluid inside the chamber does not increase by more than 0.2 atm, during the extraction of the energy by the energy-extraction device.
